(19) United States
(12) Patent Application Publication
(10) Pub. No.: US 2008/0064978 A1
(43) Pub. Date: Mar. 13, 2008

(54) System and Method for Measuring Skin Potential

(75) Inventor: Michel Bouchoucha, Paris (FR)

Correspondence Address: YOUNG & THOMPSON, 745 SOUTH 23RD STREET, 2ND FLOOR, ARLINGTON, VA, 22202, US

(73) Assignee: Universite Rene Descartes- Paris 5, Paris Cedex 06 (FR)

(21) Appl. No.: 11/587,657

(22) PCT Filed: Apr. 28, 2005
(86) PCT No.: PCT/FR05/01059

§ 371 Date: Oct. 26, 2006

(30) Foreign Application Priority Data
Apr. 28, 2004 (FR) 0404483

(51) Int. Cl. A61B 5/04

(52) U.S. Cl. 600/547

(57) ABSTRACT

A skin potential measurement system including a plurality of measurement electrodes (3) and a data processing unit (4, 2). The electrodes and the processing unit are in wireless communication. Each electrode receives a digital identification code and transmits an analog signal indicative of a patient's measured skin potential. The processing unit shapes the analog signal prior to digitization then processing thereof.

This invention relates to a system and a method for measuring skin potential using a plurality of electrodes and a processing unit.

Generally, measurement of skin potential is used in order to quantify neuromuscular depolarization in numerous physiological examinations: fixed or ambulatory electrocardiography (ECG), electroencephalography (EGG), electro-splanchnography (Holter ECG, EGG), etc. It is also used when monitoring patients under observation. Skin potential is usually measured using several electrodes connected to recorders by cable systems. However, the use of cables is a significant constraint in ambulatory and/or lengthy examinations.

The document U.S. Pat. No. 4,441,747 is known in which a protocol is described for wireless communications between electrodes and a base unit connected to a conventional electrocardiogram monitor. This solution has a drawback in particular because it requires means for adaptation to conventional electrocardiogram monitors.

The aim of this invention is to generally simplify the procedure of electrophysiological examinations. Another aim of the invention is to reduce the cost of the equipment used for recording.

At least one of the above-mentioned aims is achieved with a system for measuring skin potential comprising a plurality of measurement electrodes and a data processing unit. According to the invention, each measurement electrode is associated with an electronic module comprising:
means for generating a potential difference between the potential measured by said measurement electrode and a reference electrode inside said electronic module,
modulation means for modulating at high frequency, for example 433 MHz, said potential difference to an analogue signal,
a first transceiver for wireless transmission of this thus-modulated analogue signal to the data processing unit.
Moreover, the data processing unit comprises a second transceiver for digital transmission of an identification code of each electronic module and for receiving said analogue signal; demodulation means for demodulating this analogue signal; and shaping means for calibrating an analogue-to-digital converter, the latter being able to convert said analogue signal before processing.

With the system according to the invention, the communication of the electrodes to the processing unit (recorder) takes place by analogue wireless communication, which considerably simplifies implementation compared to the system described in the document U.S. Pat. No. 4,441,747 where the electrodes are complex and expensive because they integrate an analogue-to-digital converter. Moreover, the analogue-to-digital conversion in the processing unit according to this invention obtains a better digital resolution because the converter is calibrated with a minimum value and a maximum value. In fact, as the amplitude of the signal can vary depending on the individual, it is sensible to carry out a calibration phase.

Advantageously, each electronic module comprises a memory space containing a unique code. It is therefore possible to identify an electrode among a group of electrodes.

According to the invention, each electronic module comprises means for comparing said unique code with a code transmitted by the processing unit, and means for activating the transmission of the skin potential measured by the associated electrode when the transmitted code corresponds to said unique code. The processing unit interrogates each electrode in turn. This processing unit can be composed of a base, carrying out the communication operations with the electrodes, and a microcomputer or a PDA electronic agenda for processing the data, but there can also be a dedicated microcomputer incorporating all of the functions of the base.

The base can comprise a microcontroller for managing the communication with the electrodes and for communicating with the microcomputer or remote PDA.

The communication between the processing unit and the remote element can take place by wireless communication via the protocols WIFI, Bluetooth, etc., or by fixed wire via the protocols RS232, USB, TCP/IP, etc. The prior art document U.S. Pat. No. 4,441,747 proposes a proprietary communications protocol, which is incompatible with the use of robust and conventional protocols such as mentioned above.

The interrogation by turns is achieved because the processing unit comprises means for generating and transmitting in a cyclic manner a code associated with each electronic module.

Advantageously, each electronic module comprises time delay means in order to maintain the first transceiver in transmission mode for a predetermined period when the transmission of the skin potential must be activated.

Similarly, the processing unit comprises time delay means for maintaining the second transceiver in transmission mode for a predetermined period during the sending of a code, and for maintaining the second transceiver in receiving mode for a predetermined period in order to receive an analogue signal from an electronic module.

According to an advantageous characteristic of the invention, each electronic module comprises a supply coil of said electronic module, said coil being charged by an electromagnetic field.

According to another feature of the invention, a method for measuring skin potential is proposed embodied in a system as described above. According to the invention, the method comprises:
a calibration phase during which the processing unit interrogates each electronic module, each electronic module transmits an analogue signal representative of a measurement of skin potential, the minimum and the maximum of the analogue signals received are stored, then these minimum and maximum values are used to calibrate the analogue-to-digital converter present in the processing unit, and
a measurement phase during which each analogue signal representative of a measurement of skin potential is digitized by said analogue-to-digital converter.

Each electronic module comprises a memory space containing a unique code. This unique code is compared to a code transmitted by the processing unit, and the transmission of the skin potential measured by the associated electrode is activated when the transmitted code corresponds to said unique code.

A time delay is introduced in order to maintain the first transceiver in transmission mode for a predetermined period when the transmission of the skin potential must be activated.

Within the processing unit, a code associated with each electronic module is generated and transmitted in a cyclic manner.

Advantageously, for each code transmission, a time delay is introduced in order to maintain the second transceiver in transmission mode for a predetermined period during the sending of a code, then a time delay is introduced in order to maintain the second transceiver in receiving mode for a predetermined period in order to receive an analogue signal from an electronic module.

Other advantages and characteristics of the invention will become apparent on examination of the detailed description of an embodiment which is in no way limitative, and the attached drawings, in which:

FIG. 1 is a general view of an application of the system according to the invention;

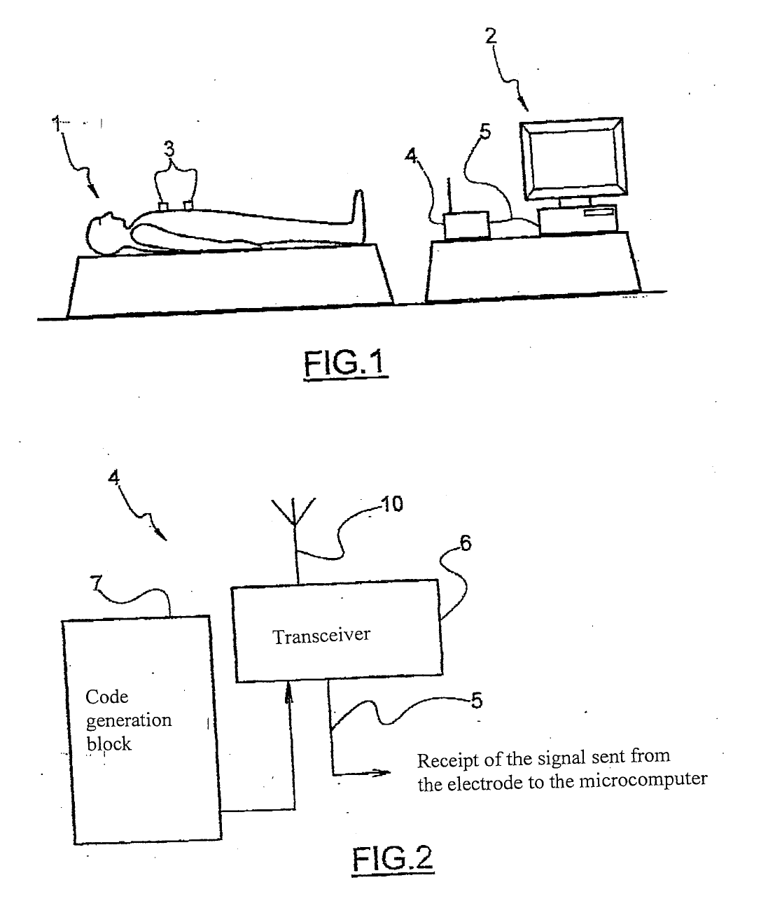

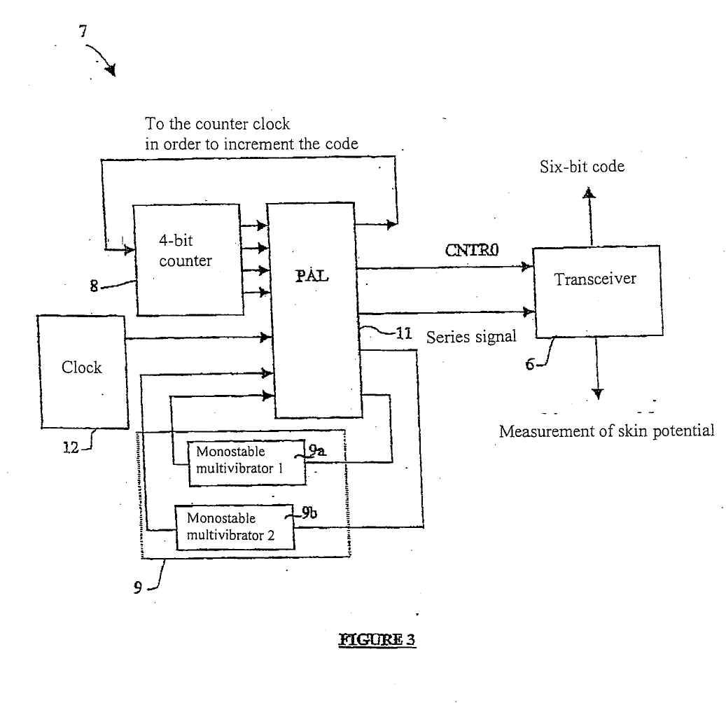

FIG. 2 is a simplified diagram illustrating the main internal elements of a base according to the invention;

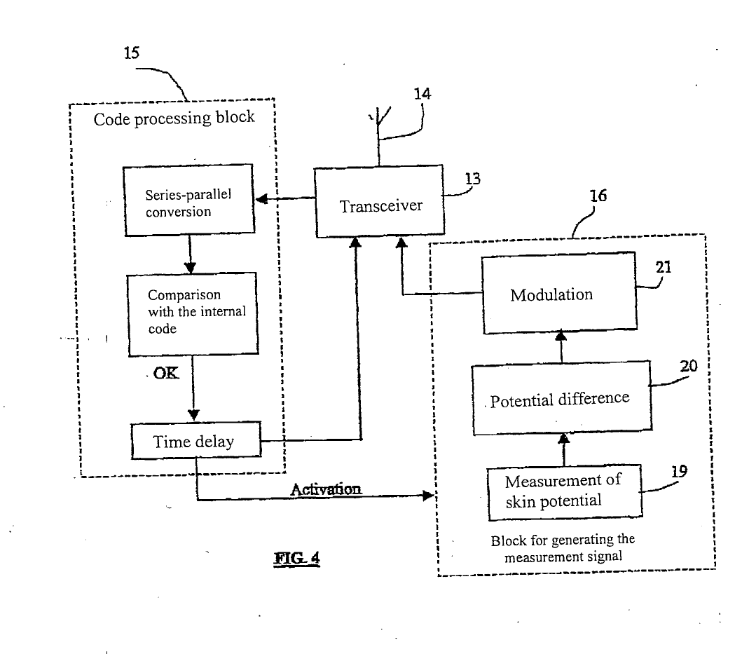

FIG. 3 is an electronic diagram illustrating the internal constitution of a code generation block according to the invention;

FIG. 4 is a simplified diagram illustrating some stages carried out in an electronic module associated with a electrode according to the invention;

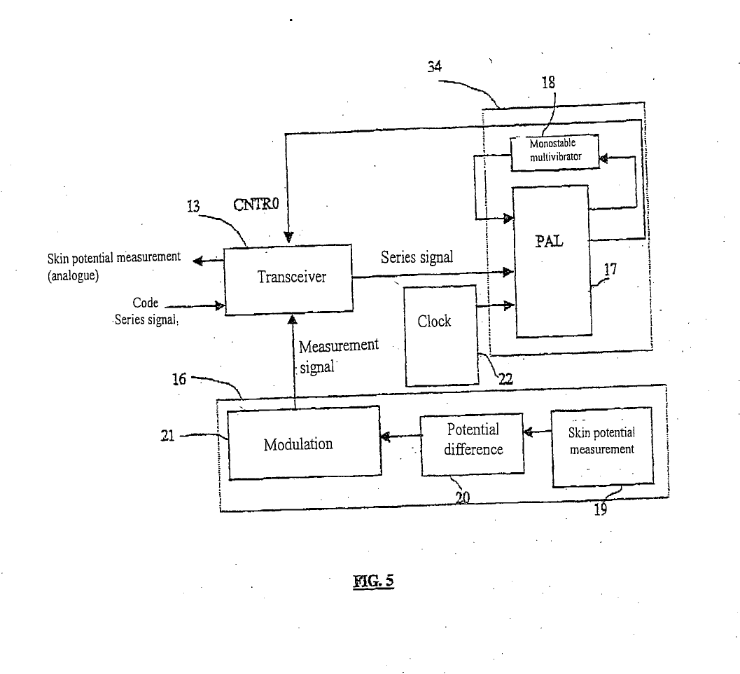

FIG. 5 is a more detailed electronic diagram illustrating the internal constitution of an electrode according to the invention;

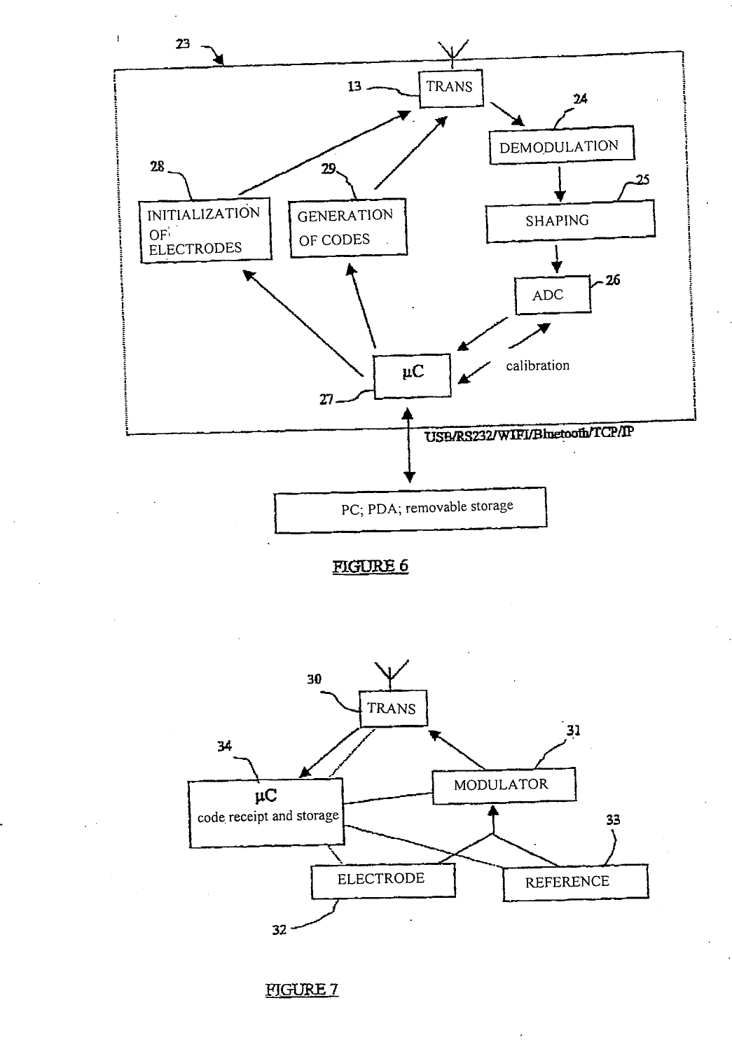

FIG. 6 is another example illustrating the main devices of a processing unit according to the invention;

FIG. 7 is another example illustrating the main devices of an electronic module according to the invention;

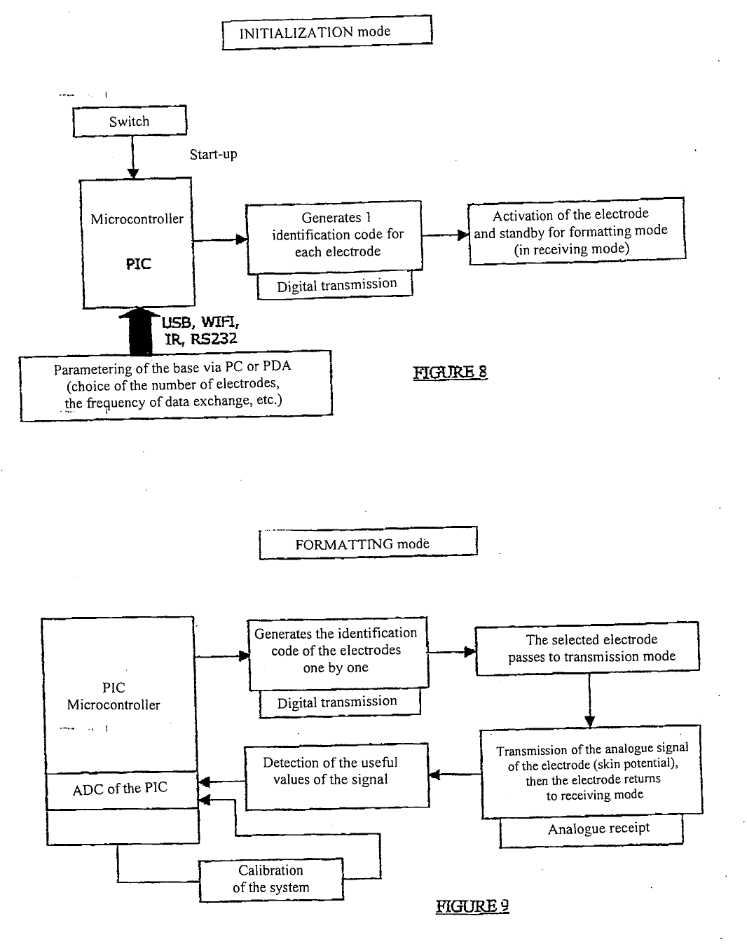

FIG. 8 is a block diagram illustrating an initialization mode according to the invention;

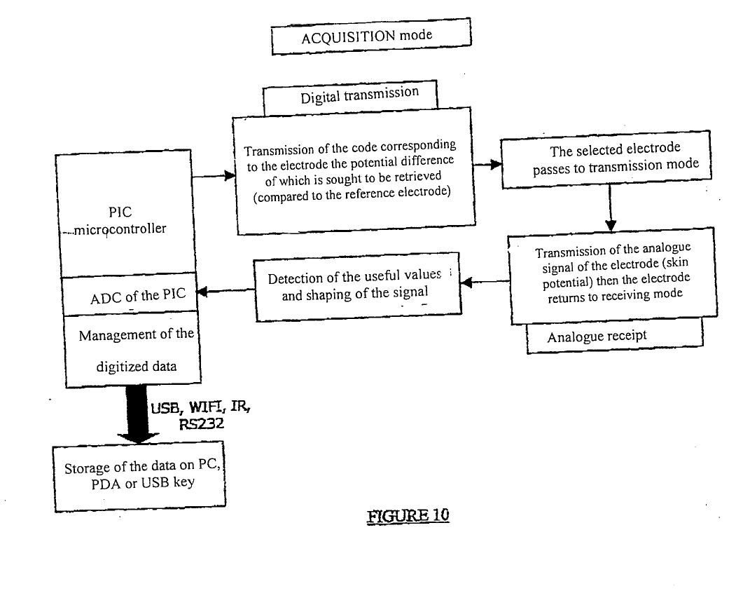

FIG. 9 is a block diagram illustrating a formatting mode according to the invention; and

FIG. 10 is a block diagram illustrating an acquisition mode according to the invention;

FIG. 1 shows a patient 1 on whom several electrodes 3 are placed according to the invention. By electrode is meant in this case a measurement electrode (or skin sensor) associated with an electronic module according to the invention. Each electrode comprises means for transmitting, by radio wave, a measurement of the skin potential of the patient 1 to a base 4. The latter can comprise means for storing the measurements received, but preferably it transmits, by fixed wire 5 or wireless link, these measurements to a microcomputer 2 serving as a recorder. It is possible to envisage the base 4 as being integrated into the microcomputer 2, this assembly constituting a processing unit.

As will be seen in more detail below, the base 4 is able to:
address an initialization signal to each electrode in an iterative manner,
receive an analogue signal, corresponding to a skin potential measurement, from an electrode, and
transmit the measurements received to the microcomputer.

In the same way each electrode comprises means for carrying out the following operations after receiving the initialization signal:
measurement of a potential difference representative of the skin potential,
modulation of the analogue signal, and
transmission of this analogue signal to the base 4.

Advantageously, the electrode according to the present invention can be constituted by a conventional electrode to which a removable adapter (multi-purpose system) is connected equipped with devices necessary for providing the assembly with the functionalities according to the present invention. However, the electrode according to the invention is preferably constituted by a single piece.

FIG. 2 shows in a little more detail the main constituent elements of the base 4.

The initialization signal from the base 4 to the electrodes 3 is a cyclic signal each cycle of which comprises the transmission of a six-bit code and, if appropriate, a time delay in order to receive a measurement. Each electrode comprises a specific code. The base 4 successively sends and in a cyclic manner all of the codes of the electrodes.

More precisely, the base 4 comprises a transceiver 6 equipped with an antenna 10 which is able to transmit a radio wave to the electrodes. The codes are produced in a code generation block 7. Once a code is sent, the code generation block 7 sets the transceiver 6 to the receive position and activates a time delay during which a skin potential measurement signal is awaited. At the end of the receiving period, the code generation block 7 resets the transceiver to transmission and generates the code for the following electrode.

In practice, the transceiver used can be a TR3100 transceiver which is ideal for short distance communication applications where there is a requirement for robust performance, small size, low power consumption and low cost. Its main characteristics are:

Power supply between 2.2 and 3.7 V;

Power supply other pins at between −0.3 and 4.0 V;

Consumption 7 mA, 0.7 μA in “SLEEP” mode;

ASK (Amplitude Shift Keying) and OOK Modulation, ASK modulation is used;

Maximum data rates: 576 kbps (500 kbps is used);

Dimensions: 10 mm×7 mm×2 mm;

Transmission receipt change over time: 107.5 μs (max).

Receipt transmission change over time: 12 μs (max).

The table allowing the modes of the integrated circuit to be defined as a function of the pins CNTRL0 and CNTRL1 is as follows:

| CNTRL0 | CNTRL1 | MODE |
|---|---|---|
| 1 | 1 | C. Receive |
| 0 | 1 | ASK Transmit |
| 1 | 0 | OOK Transmit |
| 0 | 0 | SLEEP |

FIG. 3 shows the main constituent elements of the code generation block 7. The core of this block is a programmable logic device 11, called PAL or “Programmable Array Logic”, associated with a four-bit counter 8 for generating a four-bit code for each of the electrodes, and with a timer 9. The four-bit counter 8 is a 74ALS163 device allowing the provision of a four-bit code to the PAL 11 which is programmed to carry out the loading of this code into registers, the parallel-series conversion of the code before transmission, and the management of the timer 9, the transceiver 6 and the incrementation of the counter 8. The time delay is produced by two monostable multivibrators 9a and 9b which take into consideration the receiving time of the skin potential measurement and the receiving-transmission turnaround time of the transceiver 6. Each monostable multivibrator 9a and 9b is produced by a NE555 device, which uses the time delay launch variable as an input, and is activated on the descending edge. The actual time delay variable, which is active at the high state, is retrieved at the output. The time delay is adjusted by changing the resistance values and the capacitor values of the NE555 device.

The code generation block 7 is run by a clock 12 constituted by a quartz oscillator with 1 MHz frequency wired to a MC14013 D-edge flip-flop in order to obtain a clock signal at 500 kHz.

In other words, the PAL 11 operates according to the following principle: the clock 12 and the outputs of the counter 8 are addressed at the input and the program performs the following logic functions:
parallel-series loading with shaping of the code (start bit and end bit);
transmission of the code;
launch of the time delay in the direction of the two monostable multivibrators 9a and 9b, then activation of the transceiver 6 in receiving mode; activation of the incrementation of the counter 8; and
at the end of the first time delay, the transceiver 6 is activated in transmission mode; then at the end of the second time delay, a new cycle begins.

One example of programming of the PAL 11 is given in Annex 1.

FIG. 4 shows the constituent blocks of an electrode 3. A transceiver 13 associated with an antenna 14 is seen, these elements being identical to those used in the base 4. Under normal conditions, inoperative, the transceiver 13 is in receiving mode. When a code is received, the latter is transmitted to a code processing block 15 the role of which is to perform a series-parallel conversion of the code received, a comparison of this code with the internal code of the electrode in question, then an activation (when the two codes are identical) of a block 16 generating the skin potential measurement signal. At the same time as the activation, a time delay is triggered in order to set the transceiver to transmission mode for a predetermined period. The block 16 samples an analogue signal originating from a skin sensor 19 and corresponding to the skin potential measurement. A potential difference 20 is deduced from this which is then modulated at 21 on a 433 MHz carrier for example. This modulated analogue signal is then sent to the base 4 via the transceiver 13.

In more detail in FIG. 5, the code processing block 34 can comprise a PAL 17 run by a clock 22 similar to that used for the base 4. The time delay is obtained by a monostable multivibrator 18, an NE555 device, for the transmission. The PAL 17 receives, from the transceiver 13, the series signal, i.e. the code transmitted by the base 4. The clock signal 22, the output of the monostable multivibrator 18 and the series signal received are addressed at the input of the PAL 17 which performs the following logic functions:
series-parallel loading into registers;
comparison between the loaded code and the internal code; during this time, the transceiver 13 is activated in transmission mode;
if the code does not correspond, the transceiver is reset to receiving mode;
if the code corresponds, the monostable multivibrator 18 is activated;
when the time delay ends, the transceiver 13 passes to receiving mode;
when the monostable multivibrator 18 is activated, the block 16 for generation of the measurement signal is called upon in order to allow the transmission of the measurement.

One example of programming of the PAL 17 is given in Annex 2.

FIG. 6 is another embodiment of the processing unit. The base 23 can communicate with a PC, a PDA or a removable storage device. The base 23 comprises a transceiver 13 which is able to receive the analogue signal originating from an electrode according to the invention. This analogue signal is then demodulated by the demodulator 24. This signal is then shaped by a module 25. In fact, in order that the measurement signal can be subsequently digitized, it must be shaped, i.e. the signal must be comprised between 0 and 3 V. An OFFSET of 1.5 V is added to the signal in order to raise it by means of an operational amplifier OPA (not represented). Its amplitude is also reduced in order that it does not overload the OPA. Accordingly a differentially mounted OPA is used. On the other hand, the OPA must not add any OFFSET or noise to the signal, thus the OP193 OPA is chosen.

At the output of the shaping module 25, in FIG. 6, the measurement signal is digitized by an analogue-to-digital converter ADC 26, a ten-bit, TLV 1549 with serial control, allowing the sampled signal to be transmitted. This ADC 26 is optimized during a calibration stage so as to obtain an optimal digital resolution.

A microcontroller 27 manages all of the base devices. Among other things it allows a method of initialization 28 of the electrodes and a code generation method 29 (identical to that described above) to be carried out of

FIG. 7 shows another example of embodiment of an electronic module according to the invention. Seen from the outside, the function of the transceiver 30 is to receive a digital identification code transmitted by the base, and to transmit an analogue signal representative of the skin potential measured on a patient. Seen from the inside, the transceiver 30 receives, as seen previously, an analogue signal modulated by the modulator 31. This modulator receives a signal representing a potential difference between an actual measurement electrode 32 and a reference 33. The function of the microcontroller 34 is to manage all of the devices of the electronic module, to receive and to store the identification code.

FIG. 8 shows an initialization mode with the following elements:
A switch:

Allows the start-up of the system, it is set up using a push button and positioned on a pin of one of the input/output ports of the microcontroller.

Moreover, a coil placed on the base allows each electrode to be activated when the latter is brought closer to the coil.
Parametering of the base:

A program (graphic interface) allows the configuration of the microcontroller, it is run in a computer or a PDA and allows parameters to be sent to the base via one of these communications modules (PC, PDA, etc.).
Parametering of the microcontroller:

selection of the number of electrodes to be controlled, of the communication frequency, etc.
Identification code:

Codes generated by the microcontroller [sent] to the digital transmitter, each code corresponds to 1 electrode and this allows selection of the desired electrode for the following modes.
Activation of the electrodes:

Upon receipt of their identification code, the electrodes are activated one after the other and pass to receiving mode (standby for formatting mode).

The formatting mode is described briefly in FIG. 9:
Selection of the desired electrode: transmission of the code corresponding to the desired electrode by the microcontroller via the digital transmitter, the latter is selected, passes to transmission mode and prepares to transmit the analogue signal to the receiver during the desired period of time.
Data transmission: the electrode transmits the analogue signal (difference of potential with the reference electrode) via the analogue receiver of the base to a “peak detector” block (demodulation).
Signal processing (normalization): the expected signal is sinusoidal, with a low frequency and amplitude: in order to be able to process this signal using a microcontroller, it is necessary to digitize the signal (ADC of the microcontroller), the signal must therefore be normalized (0-5 V), so as to optimize the digitization. For this purpose an electronic step is added allowing this function to be performed.
Calibration of the system: allows configuration of the ADC of the microcontroller so as to correctly digitize the signal delivered by the electrodes (number of conversion points, etc.). The signal is received from each electrode so as to calibrate the base for the storage of the data (acquisition mode).

The acquisition mode is described in FIG. 10:
This operating mode is similar to the formatting mode, a block for processing and storing the data is added.
The storage can be carried out in a PC, a PDA or using different communications modules.

In order to simplify operation the same digital transceiver (RFM) is used for the analogue and digital signals, the analogue signal must therefore be modulated before the transmission (VFC) and demodulated after receipt (FVC).

For the base, a microcontroller is chosen which has an integrated ADC, for this it is proposed to use a PIC (16F877) microcontroller.

In order to manage the electrodes, a few input/output pins and a small memory are required, one PIC microcontroller corresponding to these characteristics is used for each electrode (16F873).

Of course, the invention is not limited to the examples which have just been described and numerous adjustments can be made to these examples without exceeding the scope of the invention.

ANNEXE 1

library ieee;
use ieee.std_logic_1164.all;
usework.std_arith.all;
ENTITY ambulatoire IS

PORT ( code : in std_logic_vector(5 downto 0);

clk, tempo, recept : in std_logic ;
s, lanc_tempo, cntr0 : out std_logic );

END ambulatoire;
ARCHITECTURE archi OF ambulatoire IS
signal bascule : std_logic_vector(5 downto 0);
BEGIN
s<=bascule(0);
PROCESS(clk)
BEGIN

if (clk′event and clk = ‘1’) then

| | |
|---|---|
| if (bascule=“000000” and recept=‘0’ and tempo=‘0’) then | -- code loading |

bascule<=code;
cntr0<=‘0’;
lanc_tempo<=‘1’;

elsif (tempo=‘0’and recept=‘0’) then

| | |
|---|---|
| if(bascule=“000001”) then | -- tempo starting |

bascule(0)<=bascule(1);
cntr0<=‘1’;
lanc_tempo<=‘0’;

| | |
|---|---|
| else | -- serial/parallel loading |

bascule(0)<=bascule(1);
bascule(1)<=bascule(2);
bascule(2)<=bascule(3);
bascule(3)<=bascule(4);
bascule(4)<=bascule(5);
bascule(5)<=‘0’;
lanc_tempo<=‘1’;
cntr0<=‘0’;

end if;

elsif (bascule=“000000” and tempo=‘1’ and recept=‘1’) then-- reception during tempo

cntr0<=‘1’;
lanc_tempo<=‘1’;

elsif (bascule=“000000” and tempo=‘1’ and recept=‘0’) then

cntr0<=‘0’;
lanc_tempo<=‘1’;

end if;

end if;
END PROCESS;
END archi;

1. System for measuring skin potential comprising a plurality of measurement electrodes (3) and a data processing unit (4, 2), characterized in that each measurement electrode is associated with an electronic module comprising:
means for generating a potential difference between the potential measured by said measurement electrode and a reference electrode inside said electronic module,
modulation means for modulating said potential difference to an analogue signal,
a first transceiver (13) for wireless transmission of this thus-modulated analogue signal to the data processing unit (4, 2),
and in that the data processing unit comprises a second transceiver (10) for digital transmission of an identification code of each electronic module and for receiving said analogue signal; demodulation means for demodulating this analogue signal; and shaping means for calibrating an analogue-to-digital converter, the latter being able to convert said analogue signal before processing.

2. System according to claim 1, characterized in that each electronic module comprises a memory space containing a unique code.

3. System according to claim 2, characterized in that each electronic module comprises means for comparing said unique code with a code transmitted by the processing unit, and means for activating the transmission of the skin potential measured by the associated electrode when the transmitted code corresponds to said unique code.

4. System according to claim 3, characterized in that each electronic module comprises a time delay means in order to maintain the first transceiver in transmission mode for a predetermined period when the transmission of the skin potential must be activated.

5. System according to claim 1, characterized in that the processing unit comprises means for generating and transmitting in a cyclic manner the code associated with each electronic module.

6. System according to claim 5, characterized in that the processing unit comprises time delay means for maintaining the second transceiver in transmission mode for a predetermined period during the sending of a code, and for maintaining the second transceiver in receiving mode for a predetermined period in order to receive an analogue signal from an electronic module.

7. System according to claim 1, characterized in that the processing unit comprises a microcontroller for managing the communication with the electrodes and for communicating with a remote microcomputer.

8. System according to claim 1, characterized in that the processing unit comprises a microcontroller for managing the communication with the electrodes and for communicating with a remote PDA electronic agenda.

9. System according to claim 7, characterized in that the communication between the processing unit and the remote element takes place wirelessly via the WIFI protocol.

10. System according to claim 1, characterized in that each electronic module comprises a supply coil of said electronic module, said coil being charged by an electromagnetic field.

11. Method for measuring skin potential embodied in a system according to claim 1, characterized in that it comprises:
a calibration phase during which the processing unit interrogates each electronic module, each electronic module transmits an analogue signal representative of a measurement of skin potential, the minimum and the maximum of the analogue signals received are stored, then these minimum and maximum values are used to calibrate the analogue-to-digital converter present in the processing unit, and
a phase of measurement during which each analogue signal representative of a measurement of skin potential is digitized by said analogue-to-digital converter.

12. Method according to claim 11, characterized in that each electronic module comprises a memory space containing a unique code, this unique code is compared to a code transmitted by the processing unit, and the transmission of the skin potential measured by the associated electrode is activated when the transmitted code corresponds to said unique code.

13. Method according to claim 12, characterized in that a time delay is introduced in order to maintain the first transceiver in transmission mode during a predetermined period when the transmission of the skin potential must be activated.

14. Method according to claim 11, characterized in that, within the processing unit a code associated with each electronic module is generated and transmitted in a cyclic manner.

15. Method according to claim 14, characterized in that for each code transmission, a time delay is introduced in order to maintain the second transceiver in transmission mode for a predetermined period during the transmission of a code, then a time delay is introduced in order to maintain the second transceiver in receiving mode for a predetermined period in order to receive an analogue signal from an electronic module.

16. Method according to claim 12, characterized in that, within the processing unit a code associated with each electronic module is generated and transmitted in a cyclic manner.

17. Method according to claim 13, characterized in that, within the processing unit a code associated with each electronic module is generated and transmitted in a cyclic manner.

18. System according to claim 8, characterized in that the communication between the processing unit and the remote element takes place wirelessly via the WIFI protocol.
